(19) United States
(12) Patent Application Publication
(10) Pub. No.: US 2007/0042400 A1
(43) Pub. Date: Feb. 22, 2007

(54) Methods of preparing nucleic acid for detection

(76) Inventors: K. Yeon Choi, St. Paul, MN (US); Michael S. Zwick, Vacaville, CA (US); Kent Mccue, El Cerrito, CA (US)

Correspondence Address: BRINKS HOFER GILSON & LIONE, P.O. BOX 10395, CHICAGO, IL, 60610, US

(21) Appl. No.: 11/429,746

(22) Filed: May 8, 2006

Related U.S. Application Data
(63) Continuation of application No. PCT/US04/37488, filed on Nov. 10, 2004.
(60) Provisional application No. 60/518,895, filed on Nov. 10, 2003.
(60) Provisional application No. 60/556,584, filed on Mar. 25, 2004.

(51) Int. Cl. C12Q 1/68; C12N 1/08

(52) U.S. Cl. 435/6; 435/270

(57) ABSTRACT

Methods of preparing nucleic acid from polysaccharide-containing samples for detection by providing one or more glycosidases to the sample to degrade polysaccharides are provided. The nucleic acids can further be extracted from the sample. The method is particularly useful for detecting nucleic acid in samples with high starch content.

### RELATED APPLICATIONS

The present application claims benefit of U.S. Provisional Application Nos. 60/518,895 filed Nov. 10, 2004, and 60/556,584 filed Mar. 25, 2004, each of which is hereby incorporated by reference in its entirety.

### FIELD OF THE INVENTION

The present application relates to methods and compositions for preparing nucleic acid from a polysaccharide-containing sample by providing a glycosidase to the sample.

### BACKGROUND

Current methods of detecting and manipulating nucleic acid are frequently unsuccessful due to impurities in the sample. This is a particular problem in samples that have high polysaccharide content (such as starch-containing samples). These problems are exacerbated in samples that contain very low quantities of nucleic acid.

Preparing nucleic acid in polysaccharide-containing samples is particularly important when detecting or manipulating nucleic acid in food samples and pathogens. Such samples frequently include genetically modified organisms (GMOs), or test for product integrity or pathogenic contaminants. Correct identification of GMOs, pathogenic or other contaminants or product identity by nucleotide based methods requires that sufficient quantities of nucleic acid are obtained in sufficient purity for detection and manipulation. Conventional methods do not allow nucleic acid containing polysaccharides to be detected in very low quantities.

In addition, conventional methods of purifying nucleic acid from polysaccharide containing samples frequently use highly toxic chemicals, such as guanidine thiocyanate (GuSCN) as a toxic chaotropic salt. Such toxic contaminants can inhibit downstream manipulation of the nucleic acid. There is a tremendous need for methods that do not use compounds having the toxicity of conventional purification methods.

There is, thus, a widely recognized need for methods, compositions and kits to prepare nucleic acid in polysaccharide-containing samples for detection.

### SUMMARY OF THE INVENTION

To meet these needs, applicants have discovered a method of preparing nucleic acid for detection in polysaccharide-containing samples by providing one or more glycosidases to the sample to degrade the polysaccharide.

The method can further include extracting the nucleic acid from the sample after providing one or more glycosidases.

One or more glycosidases are provided to the polysaccharide-containing sample to degrade polysaccharides in the sample. The one or more glycosidases may include one or more glycoamylases, debranching enzymes, heterosaccharide degrading enzymes, or non-glucose homosaccharide degrading enzymes. The one or more glycoamylases can include an alpha-amylase, a beta-amylase, a glucan alpha 1,4-glucosidase, or a glucan alpha 1,6-glucosidase.

Extracting nucleic acid can include partially purifying, and/or isolating the nucleic acid. The extracting step may also include providing an alcohol to the sample. The alcohol may be ethanol, isopropanol, or a combination thereof.

The present application also includes methods of detecting nucleic acid in a polysaccharide containing sample. The nucleic acid is prepared by providing one or more glycosidases to the sample, and extracting the nucleic acid from the sample. The nucleic acid is then detected.

The nucleic acid may be any nucleic acid, as defined herein. For example, the nucleic acid may be deoxyribonucleic acid (DNA) or ribonucleic acid (RNA).

The polysaccharide may be starch. The sample may also be a food sample. Any food may be included in the sample. For example, the food sample may include corn, corn meal, soybeans, soy flour, wheat flour, papaya fruit, corn starch, corn flour, soy meal, corn chips, or maltodextrin. The food sample may also be a processed food sample.

The polysaccharides may be removed from the sample after providing one or more glycosidases prior to detection. Other cellular components may also be removed from the sample. Such cellular components may be cell membranes, cellular proteins, or other cellular debris. The cellular components may be removed by providing potassium acetate, sodium acetate, sodium chloride, ammonium acetate, or other salts to the sample to precipitate the cellular components.

Nucleic acid may also be removed from a sample by introducing the sample to a column. For example, the nucleic acid may be messenger ribonucleic acid (mRNA) and the column is an oligodeoxythymidine column. In another example, the nucleic acid may be extracted using sequence specific probe or primer.

The application also provides kits for preparing nucleic acid in a polysaccharide-containing sample for detection. The kits may include one or more glycosidases, and instructions for using the kit. The one or more glycosidases may be one or more glycoamylases or polysaccharide debranching enzymes. The one or more glycoamylases can include an alpha-amylase, a beta-amylase, a glucan alpha 1,4-glucosidase or a glucan alpha 1,6-glucosidase. The kit may further include potassium acetate, sodium acetate, sodium dodecyl sulfate (SDS), an alcohol such as ethanol, isopropanol, or a combination thereof. The kit may further include a column, a column containing glass beads or glass wool.

### BRIEF DESCRIPTION OF THE DRAWINGS

FIG. 1 depicts an agarose gel of PCR amplicons derived from nucleic acids obtained by the methods disclosed herein. The amplified nucleic acid is a portion of the invertase gene amplified from nucleic acid prepared from 1a) ground corn and 1b) corn starch.

FIG. 2 depicts composite of agarose gels of PCR amplicons derived from nucleic acid obtained by the methods disclosed herein. The amplicon is a portion of the rubisco gene amplified from nucleic acid prepared from 2a) maltodextrin, 2b) wheat flour, 2c) corn chips, 2d) corn meal, 2e) soy flour, 2f) corn kernel, and 2g) papaya fruit.

FIG. 3 depicts an agarose gel of PCR amplicons derived from nucleic acid obtained by the methods disclosed herein. The amplified nucleic acid is a portion of the lectin gene amplified from nucleic acid prepared from 3a) soy meal and 3b) soy flour, and a portion of the rubisco gene amplified from nucleic acid extracted from 3c) corn meal, and 3d) corn flour.

FIG. 4 depicts an agarose gel of PCR amplicons derived from nucleic acid obtained by the methods disclosed herein. The amplified nucleic acid is a portion of the rubisco gene amplified from nucleic acid prepared from 4a) ground corn treated with glycoamylase, 4b) corn chips treated with glycoamylase, 4c) corn starch treated with glycoamylase, 4d) ground corn not treated with glycoamylase, 4e) corn chips not treated with glycoamylase, 4f) corn starch not treated with glycoamylase, 4g) Twix® cookie treated with glycoamylase, 4h) wheat cracker treated with glycoamylase, 4i) miso power treated with glycoamylase, 4j) oat cereal treated with glycoamylase, 4k) Twix® cookie not treated with glycoamylase, 4l) wheat cracker not treated with glycoamylase, 4m) miso power not treated with glycoamylase, 4n) oat cereal not treated with glycoamylase, 4o) positive PCR control, and 4p) negative PCR control.

### DETAILED DESCRIPTION

The present patent application is directed to methods of preparing nucleic acids from a polysaccharide-containing sample for detection, as well as kits.

General Techniques

Practice of the present application employs, unless otherwise indicated, conventional techniques of molecular biology (including recombinant techniques), microbiology, cell biology, biochemistry, immunology, protein kinetics, and mass spectroscopy, which are within the skill of the art. Such techniques are explained fully in the literature, such as, Molecular Cloning: A Laboratory Manual, second edition (Sambrook and Russell, 2001) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mamnmalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); and Short Protocols in Molecular Biology (Wiley and Sons, 1999); all of which are incorporated herein by reference in their entirety. Furthermore, procedures employing commercially available assay kits and reagents typically are used according to manufacturer-defined protocols unless otherwise noted.

### DEFINITIONS

“Sample” refers to, but is not limited to, a liquid sample of any type (e.g. water, a buffer, a solution, or a suspension), or a solid sample of any type (e.g. cells, food, water, air, dirt, grain, or seed), and combinations thereof.

“Nucleic acid” refers to a chain of nucleic acid of any length, including deoxyribonucleotides (DNA), ribonucleotides (RNA), or analogs thereof. A nucleic acid may have any three-dimensional structure, and may perform any function, known or unknown. The following are non-limiting examples of nucleic acid: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A nucleic acid may include modified nucleotides, such as methylated nucleotides and nucleotide analogs. If present, modifications to the nucleotide structure may be imparted before or after assembly of a nucleic acid polymer. The sequence of a nucleic acid may be interrupted by non-nucleotide components. A nucleic acid may be further modified after polymerization, such as by conjugation with a labeling component.

“Polysaccharide” refers to any combination of monosaccharide or monosaccharide derivatives covalently linked together into linear or branched chains. The polysaccharide may be a homopolysaccharide (including only one type of monosaccharide), or a heterosaccharide (including two or more types of monosaccharide). Starch is an example of a polysaccharide. As used herein, “polysaccharide” and “oligosaccharide” are used interchangeably.

“Glycosidase” refers to any polysaccharide-degrading enzyme. “Degrading” refers to breaking one or more bonds between monosaccharide or monosaccharide derivative units the polysaccharide.

“Glycoamylase” refers to any enzyme that hydrolyzes glycosyl bonds in glucose homopolysaccharides. As used herein, glycoamylase includes alpha-amylases, beta-amylases, glucan alpha 1,4-glucosidases, and glucan alpha 1,6-glucosidases.

“Extracting” refers to removing one or more classes of compounds from a sample. For example, “extracting” can include introducing an alcohol to the sample, column based purification, or sequence specific hybridization.

“Partially Purify” refers to removing one or more compounds or classes of compounds from a mixture of compounds or mixture of classes of compounds. For example, “partially purifying nucleic acids” refers to removing one or more nucleic acids from a mixture of nucleic acids and non-nucleic acids. Partially purified compounds may be accompanied by additional compounds.

“Isolate” refers to separating one compound or class of compounds from a mixture of compounds or class of compounds. For example, “isolating nucleic acid” refers to removing one nucleic acid from a mixture of nucleic acid and non-nucleic acid components.

“High starch content” refers to samples that contain greater than about 60% starch or complex carbohydrate by weight. Examples of samples having a “high starch content” include, but are not limited to, flour, grain, grain meal, potato and other tuber samples. Other examples may include blends of high starch compounds in processed food products such as breakfast cereals.

Methods of Preparing Nucleic Acid

A method of preparing nucleic acid from a polysaccharide containing sample for detection is provided. One or more glycosidases are added to the polysaccharide-containing sample to degrade polysaccharides therein. The nucleic acid may then be extracted. The nucleic acid may then be detected, amplified, identified by hybridization-based method, or otherwise manipulated.

In conventional methods of preparing nucleic acid, polysaccharides such as starch often co-precipitate with nucleic acid. When polysaccharides co-precipitate with nucleic acid, it is difficult to manipulate nucleic acids by amplification methods, such as PCR, or by other detection methods, such as hybridization detection. Polysaccharides may also inhibit digestion with restriction endonucleases and other enzymatic manipulations. When polysaccharides are degraded by glycosidases by the methods of the present application, the nucleic acid may be readily detected, amplified or digested.

Glycosidases

Glycosidases may be, for example, glycoamylases, debranching enzymes, heterosaccharide degrading enzymes, or non-glucose homopolysaccharide degrading enzymes.

Glycoamylase

Glycoamylase is used to degrade polysaccharides in a sample containing nucleic acid. As used herein, “glycoamylase” includes any enzyme that hydrolyzes glycosyl bonds in polysaccharides. Glycoamylases include alpha-amylases, beta-amylases, glucan alpha 1,4-glucosidases, and glucan alpha 1,6-glucosidases.

Alpha-amylases are enzymes that are involved in the endohydrolysis of 1,4-alpha-glucosidic linkages in oligosaccharides and polysaccharides. This enzyme is also known as 1,4-alpha-D-glucan glucanohydrolase and glycogenase. The enzyme acts on starch, glycogen and related polysaccharides and oligosaccharides. Examples of alpha-amylases may be found, for example, at the website of the Biomolecular Structure and Modeling Group, Department of Biochemistry and Molecular Biology, University College, London. Other examples are discussed, for example, in Sauer J, Sigurskjold B W, Christensen U, Frandsen T P, Mirgorodskaya E, Harrison M, Roepstorff P, Svensson B., Glycoamylase: structure/function relationships, and protein engineering, Biochem Biophys Acta. Dec. 29, 2000; 1543(2):275-293, and Coutinho P M, Reilly P J., Structure-function relationships in the catalytic and starch binding domains of glycoamylase, Protein Eng. March 1994;7(3):393-400.

Beta-amylases are enzymes that are involved in hydrolysis of 1,4-alpha-glucosidic linkages in polysaccharides so as to remove successive maltose units from the non-reducing ends of the chains. The enzymes are also known as 1,4-alpha-D-glucan maltohydrolase, saccharogen amylase, or glycogenase. Beta-amylases act on starch, glycogen and related polysaccharides and oligosaccharides producing beta-maltose by an inversion. Examples of beta-amylases may be found, for example, at the website of the Biomolecular Structure and Modeling Group, Department of Biochemistry and Molecular Biology, University College, London. Other examples are discussed, for example, in Sauer J. Sigurskjold, Christensen Frandsen, Mirgorodskaya Harrison, Roepstorff Svensson, Glycoamylase: structure/function relationships, and protein engineering, Biochem Biophys Acta. Dec. 29, 2000; 1543(2):275-293, and Coutinho Reilly, Structure-function relationships in the catalytic and starch binding domains of glycoamylase, Protein Eng. March 1994;7(3):393-400.

Glucan alpha 1,4-glucosidase is an enzyme involved in the hydrolysis of terminal 1,4-linked alpha-D-glucose residues successively from non-reducing ends of the chains with release of beta-D-glucose. The enzyme is also known as glucoamylase, 1,4-alpha-D-glucan glucohydrolase, amyloglucosidase, gamma-amylase, lysosomal alpha-glucosidase, and exo-1,4-alpha-glucosidase. Some forms of this enzyme can rapidly hydrolyze 1,6-alpha-D-glucosidic bonds when the next bond in sequence is 1,4-, and some preparations of this enzyme hydrolyze 1,6- and 1,3-alpha-D-glucosidic bonds in other polysaccharides. Examples of glucan alpha 1,4-glucosidases may be found, for example, at the website of the Biomolecular Structure and Modeling Group, Department of Biochemistry and Molecular Biology, University College, London. Other examples are discussed, for example, in Sauer Sigurskjold, Christensen Frandsen, Mirgorodskaya Harrison, Roepstorff Svensson, Glucoamylase: structure/function relationships, and protein engineering, Biochem Biophys Acta. Dec. 29, 2000; 1543(2):275-293, and Coutinho Reilly, Structure-function relationships in the catalytic and starch binding domains of glucoamylase, Protein Eng. March 1994;7(3):393-400.

Polysaccharide De-branching Enzymes

Polysaccharide debranching enzymes cleave the α-1,6 bond in polysaccarides. Polysaccharide debranching enzymes include any debranching enzyme known in the art. Debranching enzymes include two general categories: isoamylases and pullulanases (such as R-enzymes). Pullalanases can hydrolyze the α1,6-linkages in polysaccarides. These molecules include the yeast glucan pullulan R-enzymes, and are discussed, for example, in Nakamura Y, Umemoto T, Ogata N, Kuboki Y, Yano M, Sasaki T (1996); Starch debranching enzyme (R-enzyme or pullulanase) from developing rice endosperm: purification, cDNA and chromosomal localization of the gene; Planta 199: 209-218, Nakamura Y. Umemoto T. Takahata Y. Komae K. Amano E. Satoh H (1996); and Changes in structure of starch and enzyme activities affected by sugary mutations in developing rice endosperm: possible role of starch debranching enzyme (R-enzyme) in amylopectin biosynthesis. Physiol Plant 97: 491 498).

Heterosaccharide and Non-glucose Homosaccharide Degrading Enzymes

Glycosidases also include heterosaccharide degrading enzymes and non-glucose homopolysacchardide degrading enzymes. These enzymes may include any heterosaccharide degrading enzyme or a non-glucose homopolysacchardide degrading enzyme known in the art. Heterosaccharide degrading enzymes include, but are not limited to, xylosidases. Non-glucose or a non-glucose homopolysacchardide degrading enzymes include, for example, glycuronidases.

Glycosidases may be obtained from a variety of sources, including bacteria, plants, and fungi, and animals. Examples of bacterial sources include, but are not limited to, Bacillus (such as Bacillus subtilis, Bacillus licheniformis, Bacillus amyloliquefaciens, and Bacillus stearothermophilus), Streptomyces (such as Streptomyces tendae) Thermoanaerobacteria, Alteromonas haloplanktis, and Pseudoalteromonas haloplanctis. Examples of fungal sources include, but are not limited to, Aspergillus niger, Aspergillus oryzae, Aspergillus sp. and Rhizopus sp. Examples of plant sources include, but are not limited to, Barley seeds (Hordeum vulgare) Amaranthus hypochondriacus (prince's feather), and Phaseolus vulgaris (kidney bean). Animal sources include, but are not limited to, mammals, including humans.

Glycosidases may also be acquired commercially. For example, amyloglucosidase from Aspergillus niger or Rhizopus sp. may be acquired from Sigma-Aldrich (St. Louis, Mo.), VWR International (Brisbane, Calif.), ICN Biomedicals (Costa Mesa, Calif.), Neogen (Lexington Ky.), and American Laboratories Inc. (Omaha, NB).

A. Providing One or More Glycosidases

In the methods of the present application, one or more glycosidases are provided to a sample to degrade polysaccharides in the sample. Glycosidases degrade polysaccharides found in the sample that would interfere with purification, detection or amplification of nucleic acid, particularly low quantities of nucleic acid.

Low quantities of nucleic acid may be less than about 1000 ng, less than about 500 ng, less than about 400 ng, less than about 300 ng, less than about 200 ng, less than about 100 ng, less than about 5 ng, or less than about 0.1 ng. Extracting low amounts of nucleic acid from numerous competing substrates, including polysaccharides, often leaves less than 2 ng of nucleic acid per microliter which may not be enough for downstream applications.

The sample may contain at least about 10%, at least about 15%, at least about 20%, at least about 25%, at least about 30%, at least about 35%, at least about 40%, at least about 45%, at least about 55%, at least about 60%, at least about 65%, at least about 70%, at least about 75%, at least about 80%, at least about 85%, at least about 90%, at least about 95%, or at least about 100% polysaccharide by weight. When the sample contains at least about 100% polysaccharide by weight, nucleic acid is present in trace amounts.

The one more glycosidases may include one or more glycoamylases, debranching enzymes, heterosaccharide degrading enzymes, and non-glucose homopolysaccharide degrading enzymes.

The polysaccharide may be degraded by one or more glycoamylases. In particular, the polysaccharide degraded by the glycoamylase is starch. Starch is the nutritional reservoir found in plants, and is a polymeric glucose chain. Starch occurs in two forms: amylose, which contains solely α-1,4 linkages of glucose monomers, and amylopectin, a branched form containing about one α-1,6 glucose-glucose linkage per every 30 α-1,4 glucose-glucose linkages. By degrading starch present in a sample, nucleic acid may be detected.

Glycosidases may be provided in the form of a liquid solution. The glycosidase may be provided at any concentration. The greater the concentration of polysaccharides in a sample, the greater the concentration of glycosidase that needs to be added. For example, one unit will produce 10 mg of glucose from a buffered 1% starch solution in 30 minutes at 40° C. One unit will dextrinize 1 mg of starch per minute at pH 6.6 and 30° C. At 50 U, 500 mg of polysaccharide is degraded in 10 minutes. For example, 50 U of enzyme per mL of solution nucleic acid containing solution degrades polysaccharides sufficiently to detect polynucleotides.

The one or more glycosidases may be added in combination with a solution that precipitates saccharides, such as potassium acetate or sodium acetate. Alternatively one or more glycosidases may be added before the salts to avoid precipitation by high salt concentrations. The glycosidase reaction may be heated to increase the rate of polysaccharide degradation.

The sample includes materials suspected to contain biological entities. It need not be limited as regards to the source of the sample or the manner in which it is made. Generally, the sample can be biological and/or environmental samples. Biological samples may be derived from human or other animals, body fluid, solid tissue samples, tissue cultures or cells derived therefrom and the progeny thereof, sections or smears prepared from any of these sources, or any other samples that contain nucleic acid. Preferred biological samples are body fluids including but not limited to urine, blood, cerebrospinal fluid (CSF), sinovial fluid, semen, ammoniac fluid, and saliva. Other types of biological sample may include food products and ingredients such as cereals, flours, dairy items, vegetables, meat and meat by-products, and waste. Environmental samples are derived from environmental material including but not limited to soil, water, sewage, cosmetic, agricultural and industrial samples, as well as samples obtained from food and dairy processing instruments, apparatus, equipment, disposable, and non-disposable items.

In one embodiment, the samples are high starch containing samples. Examples of samples having a “high starch content” include, but are not limited to, flour, grain, grain meal, starch, sugar, potato and other tuber samples. Other examples may include blends of high starch compounds in processed food products such as breakfast cereals. Starch containing samples include processed foods, corn, corn meal, soybeans, soy flour, wheat flour, papaya fruit, and corn starch. Processed foods can include corn-containing foods, such as commercially available breakfast cereals and corn chips.

The sample may be in solid form, liquid form, gel form or as a suspension. In some instance, a solid sample may be ground prior to providing glycosidase. The sample may take the form of a suspension, or may be solubilized by one or more solvents.

The methods disclosed herein also may include removing additional non-nucleic acid components from the sample before or after the glycolamylase is administered. Cells may be lysed and non-nucleic acid material may be removed using methods well known in the art. For example, and proteins denatured by treating the sample with a detergent such as sodium dodecyl sulfate (SDS). Other methods may be found, for example, in Sambrook H. and Russell, 2001 Molecular Cloning: A Laboratory Manual, 3rd ed Cold Spring Harbor Press, Cold Spring Harbor, N.Y.; Permingeat H R, Romagnoli M V, and Vallejos R H, 1998, A simple method for isolating high yield and quality DNA from cotton (G. hirsutum L.) leaves; Plant Mol Biol. Reporter 16:1-6; Paterson A H, Brubaker C L and Wendel J F, 1993, A rapid method for extraction of cotton (Gossypium spp.) genomic DNA suitable for RFLP or PCR analysis; Plant Mol Biol. Reporter 11(2) 122-127; and Couch J A and Fritz P. 1990, Extraction of DNA from plants high in polyphenolics; Plant Mol Biol. Reporter 8(1) 8-12.

Proteins and peptides may be also removed by methods known in the art. Potassium acetate or sodium acetate, for example, may be used to precipitate carbohydrates and proteins prior to extracting nucleic acid. Potassium acetate and sodium acetate also aid in the precipitation of proteins and carbohydrates out of the solution and thus leaves the nucleic acid free to bind to glass particles during nucleic acid extraction. In another example, proteins and peptides may be removed by phenol extraction, and denatured using of detergents such as sodium dodecyl sulfate (SDS) in a suitable buffer such as Tris-EDTA. Samples may be heated during this process, and centrifuged to remove non-nucleic acid components. The non-nucleic acid solid material may be removed via centrifugation, optionally after heating.

If the nucleic acid is a ribonucleotide (RNA) molecule, then degradation of RNA may be reduced or minimized by removing RNA nucleases. RNA degradation may be prevented by well-known methods such as adding proteases to degrade RNases that remain in the sample. For example, RNase free proteinase, may be added. Alternatively inhibitors of RNase may be added such as PNAsin. See, for example, Sambrook, J., Russell, D. W., Molecular Cloning: A Laboratory Manual, the third edition, Cold Spring Harbor Laboratory Press, Cold Spring Harbor, N.Y., 7.82, 2001.

B. Extracting Nucleic Acid

Nucleic acid may be extracted from the resulting solution. Nucleic acid may be extracted by one or more methods known in the art.

Nucleic acid may be extracted by introducing solvents, often in the presence of salts that precipitate nucleic acid to the sample. For example, the nucleic acid may be extracted by being placed in an alcohol solution, such as an ethanol or isopropanol solution. Any concentration of alcohol may be provided. For example, a solution of at least about 75%, 80%, 85%, 90%, or 95% ethanol may be provided to a sample to extract the nucleic acid. Alternatively, in another example, a solution of at least about 75%, 80%, 85%, 90%, or 95% isopropanol may be provided to a sample to extract the nucleic acid. Nucleic acid may also be precipitated by adding polyethylene glycol to the sample.

Alternatively, nucleic acid may be extracted by introducing a solvent that precipitates components other than nucleic acid. In this case, nucleic acid remains in the solution and other components are removed.

The nucleic acid also may be extracted by column based purification. Column based extraction may be conducted using columns known in the art. In one embodiment, the column may be glass beads. Such glass beads provide a large pore, silica bead binding matrix that may be used to alleviate clogging that commonly occurs with extractions of nucleic acid from high starch compounds and currently available silica wafer-like columns. These columns may be obtained commercially from, for example, ISC Bioexpress (Kaysville, Utah), VWR (Buffalo Grove, Ill.), Axygen (Union City, Calif.). Glass beads are then added to the column. Alternatively the bottom of a microfuge tube may be pierced with a small needle (making a hole or holes) and filled with glass beads. Alternatively glass fiber filters may be added to the column. Unlike glass milk or diatomaceous earth, the beads do not compact and therefore allow a much better flow through of the supernatant. If residual starch is present such columns do not clog and can still bind DNA efficiently.

Nucleic acid may be extracted by separating the nucleic acid via column chromatography, such as high performance liquid chromatography (HPLC) or FPLC.

Nucleic acid may also be extracted using a column that specifically binds nucleic acid. For example, glass bead columns specifically bind nucleic acid in a sample. The nucleic acid may then be eluted from the column. Other columns are known in the art.

The nucleic acid may also be extracted in a sequence specific manner. For example, a discrete nucleic acid sequence may be extracted by hybridization to an immobilized sequence specific probe. Methods of obtaining nucleic acid by hybridization methods are well known in the art, as described, for example, in Mark Schena, MicroArray Analysis, Wiley-Liss, John Wiley & Sons, Hoboken N.J. (2003). The sequence specific probe may be attached to a sold surface, such as via a biotin-avidin interaction, before or after hybridization of the probe to nucleic acid in the sample. The DNA molecules may be visualized by directly staining the amplified products with a DNA-intercalating dye. As is apparent to one skilled in the art, exemplary dyes include but not limited to SYBR green, SYBR blue, DAPI, propidium iodine, Hoeste, SYBR gold and ethidium bromide. The amount of luminescent dyes intercalated into the amplified DNA molecules is directly proportional to the amount of the amplified products, which can be conveniently quantified using a FluroImager (Molecular Dynamics) or other equivalent devices according to manufacturers' instructions. A variation of such an approach is gel electrophoresis of amplified products followed by staining and visualization of the selected intercalating dye. Alternatively, labeled oligonucleotide hybridization probes (e.g. fluorescent probes such as FRET probes and colorimetric probes) may be used to detect amplification.

RNA may be extracted using a column containing oligodeoxythymidine hybridization sequence. For example, messenger RNA (mRNA) may be extracted using an oligodeoxythymidine column. Columns may be prepared manually, or obtained commercially. Alternatively RNA may also be bound to glass beads: This is performed as with the DNA with the alteration of pH above 6.3 and high salt concentrations.

The nucleic acid may be partially purified or isolated after extraction. The nucleic acid may be partially purified or isolated using any of the extraction methods discussed above. Oligodeoxythymidine columns may be obtained commercially, for example, from Molecular Research Center Inc. (Cincinnati, Ohio), Stratagene (La Jolla, Calif.), Invitrogen (Carlsbad, Calif.), or Amersham (Pistcataway, N.J.).

Nucleic acid may also be resolubilized prior to use, typically in a buffer. Methods of resolubilization are well-known in the art as disclosed in, for example, Sambrook H, E F Fritsch and Maniatis T, 1989 Molecular Cloning: A laboratory manual 2nd ed Cold Spring Harbor Press, Cold Spring Harbor, N.Y.

If the nucleic acid is a ribonucleotide (RNA) molecule, then additional proteinases may be added to prevent degradation of the nucleic acid. For example, RNase-free proteinase K may be added to the sample to prevent the RNA from degrading.

C. Detecting Nucleic Acid

Nucleic acid may optionally be detected by any method known in the art. In particular, nucleic acid may be detected by amplification or hybridization methods.

The nucleic acid may be detected by amplification methods. For example, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, Taq polymerase, Tth polymerase, Pfu polymerase and/or RNA polymerases such as reverse transcriptase. Tth polymerase also has reverse transcriptase activity.

A preferred amplification method is PCR. General procedures for PCR are taught in U.S. Pat. No. 4,683,195 (Mullis et al.) and U.S. Pat. No. 4,683,202 (Mullis et al.). However, optimal PCR conditions used for each amplification reaction are generally empirically determined or estimated with computer software commonly employed by artisans in the field. A number of parameters influence the success of a reaction. Among them are annealing temperature and time, extension time, Mg²⁺, pH, and the relative concentration of primers, templates, and deoxyribonucleotides. Generally, the template nucleic acid is denatured by heating to at least about 95° C. for 1 to 10 minutes prior to the polymerase reaction. Approximately 20-99 cycles of amplification are executed using denaturation at a range of 90° C. to 96° C. for 0.05 to 1 minute, annealing at a temperature ranging from 48° C. to 72° C. for 0.05 to 2 minutes, and extension at 68° C. to 75° C. for at least 0.1 minute with an optimal final cycle. In one embodiment, a PCR reaction may contain about 100 ng template nucleic acid, 20 uM of upstream and downstream primers, and 0.05 to 0.5 mm dNTP of each kind, and 0.5 to 5 units of commercially available thermal stable DNA polymerases.

A variation of the conventional PCR is reverse transcription PCR reaction (RT-PCR), in which a reverse transcriptase first coverts RNA molecules to single stranded cDNA molecules, which are then employed as the template for subsequent amplification in the polymerase chain reaction. In carrying out RT-PCR, the reverse transcriptase is generally added to the reaction sample after the target nucleic acid is heat denatured. The reaction is then maintained at a suitable temperature (e.g. 30-45° C.) for a sufficient amount of time (10-60 minutes) to generate the cDNA template before the scheduled cycles of amplification take place. Alternatively, Tth DNA polymerase can be employed for RT-PCR. One of skill in the art will appreciate that if a quantitative result is desired, caution must be taken to use a method that maintains or controls for the relative copies of the amplified nucleic acid. Methods of “quantitative” amplification are well known to those of skill in the art. For example, quantitative PCR can involve simultaneously co-amplifying a known quantity of a control sequence using the same primers. This provides an internal standard that may be used to calibrate the PCR reaction.

One internal standard is a synthetic AW106 cRNA. The AW106 cRNA is combined with RNA isolated from the sample according to standard techniques known to those of skill in the art. The RNA is then reverse transcribed using a reverse transcriptase to provide cDNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

In addition to conventional PCR and RT-PCR, another preferred amplification method is ligase chain polymerase chain reaction (LCR). The method involves ligation of a pool of nucleic acids derived from a sample to a set of primer pairs, each having a target-specific portion and a short anchor sequence unrelated to the target sequences. A second set of primers containing the anchor sequence is then used to amplify the target sequences linked with the first set of primers. Procedures for conducting LCR are well known to artisans in the field, and hence are not detailed herein (see, e.g., WO 97/45559, WO 98/03673, WO 97/31256, and U.S. Pat. No. 5,494,810).

The aforementioned amplification methods are highly sensitive, amenable for large-scale identification of multiple biological entities using extremely small quantities of sample.

Nucleic acid may also be detected by hybridization methods. In these methods, labeled nucleic acid may be added to a substrate containing labeled or unlabeled nucleic acid probes. Alternatively, unlabeled or unlabeled nucleic acid may be added to a substrate containing labeled nucleic acid probes. Hybridization methods are disclosed in, for example, MicroArray Analysis, Marc Schena, John Wiley and Sons, Hoboken N.J. 2003.

Methods of detecting nucleic acids can include the use of a label. For example, radiolabels may be detected using photographic film or a phosphoimager (for detecting and quantifying radioactive phosphate incorporation). Fluorescent markers may be detected and quantified using a photodetector to detect emitted light (see U.S. Pat. No. 5,143,854, for an exemplary apparatus). Enzymatic labels are typically detected by providing the enzyme with a substrate and measuring the reaction product produced by the action of the enzyme on the substrate. Colorimetric labels are detected by simply visualizing the colored label.

In one embodiment, the amplified nucleic acid molecules are visualized by directly staining the amplified products with a nucleic acid-intercalating dye. As is apparent to one skilled in the art, exemplary dyes include but not limited to SYBR green, SYBR blue, DAPI, propidium iodine, Hoeste, SYBR gold and ethidium bromide. The amount of luminescent dyes intercalated into the amplified DNA molecules is directly proportional to the amount of the amplified products, which can be conveniently quantified using a FluroImager (Molecular Dynamics) or other equivalent devices according to manufacturers' instructions. A variation of such an approach is gel electrophoresis of amplified products followed by staining and visualization of the selected intercalating dye. Alternatively, labeled oligonucleotide hybridization probes (e.g. fluorescent probes such as fluorescent resonance energy transfer (FRET) probes and colorimetric probes) may be used to detect amplification. Where desired, a specific amplification of the genome sequences representative of the biological entity being tested, may be verified by sequencing or demonstrating that the amplified products have the predicted size, exhibit the predicted restriction digestion pattern, or hybridize to the correct cloned nucleotide sequences.

D. Devices

The methods described above may be conducted using devices known in the art.

The methods disclosed herein may be practiced using individual tubes. Samples may be transferred between tubes, or kept in the same tube during the method.

The methods disclosed herein may be practiced using a multi-site test device, such as a multi-well plate or series of connected tubes (“strip tubes”). The method may involve the steps of placing aliquots of a nucleic acid containing sample into at least two sites of a multi-site test device, and simultaneously providing one or more glycosidases in each of the sites. Samples may be manipulated between different multi-site devices, or between different sites in the same multi-site device.

The multi-site test device includes a plurality of compartments separated from each other by a physical barrier resistant to the passage of liquids and forming an area or space referred to as “test site.” The test sites contained within the device can be arrayed in a variety of ways. In a preferred embodiment, the test sites are arrayed on a multi-well plate. It typically has the size and shape of a microtiter plate having 96 wells arranged in an 8×12 format. 384 well plates may also be used. One advantage of this format is that instrumentation already exists for handling and reading assays on microtiter plates; extensive re-engineering of commercially available fluid handling devices is thus not required. The test device, however, may vary in size and configuration. It is contemplated that various formats of the test device may be used which include, but are not limited to thermocycler, lightcycler, flow or etched channel PCR, multi-well plates, tube strips, microcards, petri plates, which may contain internal dividers used to separate different media placed within the device, and the like. A variety of materials can be used for manufacturing the device employed in the present application.

In general, the material with which the device is fabricated does not interfere with amplification reaction and/or immunoassays. A preferred multi-site testing device is made from one or more of the following types of materials: (poly)tetrafluoroethylene, (poly)vinylidenedifluoride, polypropylene, and polystyrene.

The device may be the device disclosed in U.S. Pat. No. 6,626,051.

Uses for Methods of Preparing Nucleic Acid

The present methods are particularly useful for preparing nucleic acid in polysaccharide containing samples.

Starch Containing Samples

The present methods may be used to prepare nucleic acid in polysaccharide-containing samples, such as starch-containing samples particularly high starch samples, as described above. Starch containing samples include seeds, corn, corn meal, soybeans, soy flour, wheat flour, papaya fruit, and corn starch.

Food based Samples

The methods disclosed herein may also be used to prepare nucleic acid in food samples. Food based samples include prepared foods, such as corn, corn meal, soybeans, soy flour, wheat flour, papaya fruit, corn starch, corn chips and maltodextrin. Other food samples include crops and leaf tissue. Additional components, such as antioxidants, may be required for leaf tissue. The methods herein also may be used to obtain nucleic acid from meat samples. The nucleic acid may subsequently be used to identify out-of-season animals, endangered species or if material from any species (or multiple species) are present in a sample (such as peanut residue in a food product or ungulate material in cow feed).

The present methods may also be used to prepare nucleic acid from processed food samples. Food processing often includes extensive mixing and milling procedures, as well as high temperature cooking procedures. Many processed foods contain large quantities of polysaccharides, and low quantities of nucleic acids. Examples of processed foods include, but are but not limited to, oat cereals, O's cereal, crackers, dried tofu, miso powder, polenta, Twix® cookies and soynut butter.

Pathogens

The methods disclosed herein may be used to prepare nucleic acid from pathogens. Generally, the presence of a pathogen or the presence of pathogen-related nucleic acid in a host is detected by analysis of nucleic acid in a sample. Foodborne pathogens, however, are frequently contained in high polysaccharide samples, such as high starch samples. By following the methods disclosed herein, nucleic acid specific to pathogens may be detected. This requires the additional steps of disrupting the microbial cell wall and allowing the microorganim to lyse. Methods to do this are known in the art. For example, lysozyme (Sigma, St. Louis Mo.) can be used to disrupt the cell wall of gram positive bacteria. (Flamm R K, Hinrichs D J, Thomashow M F. Infect Immun. April 1984; 44(1):157-61) At low concentrations (40 ng/100 ul TE), lysomzyme can also be used to disrupt gram negative bacteria for nucleic acid isolation. For yeasts, zymolyase or lyticase (van Burik J A, Schreckhise R W, White T C, Bowden R A, Myerson D. Med Mycol. October 1998;36(5):299-303) can be used to digest the cell wall and create spheroplasts for easier nucleic acid isolation. Other buffers that can be used include 2-mercaptoethanol, sorbitol buffer, N-lauryl sarcosine sodium salt solution, sodium or potassium acetate solution.

Examples of pathogens or presence of the pathogen for which the nucleic acid may be prepared according to the present methods and assay systems include, but are not limited to, Staphylococcus epidermidis, Escherichia coli, methicillin-resistant Staphylococcus aureus (MSRA), Staphylococcus aureus, Staphylococcus hominis, Enterococcus faecalis, Pseudomonas aeruginosa, Staphylococcus capitis, Staphylococcus warneri, Klebsiella pneumoniae, Haemophilus influenzae, Staphylococcus simulans, Streptococcus pneumoniae and Candida albicans.

Nucleic acid associated with foodborne pathogens may be prepared by the methods disclosed herein. The method may be used to detect nucleic acid from Listeria, Campylobacter, E. coli and Salmonella.

Additional examples include, but are not limited to, Bacillus anthracis (Anthrax), Clostridium botulinum (Botulism), Brucellae (Brucellosis), Vibrio cholera (Cholera), Clostridium perfringens (gas gangrene, Clostridial myonecrosis, enteritis necroticans), Ebola virus (Ebola Hemorrhagic Fever), Yersinia pesits (Plague), Coxiella burnetii (Q Fever), and Smallpox virus (Smallpox).

Nucleic acid having sequences specific to different pathogens may be further prepared by the nucleic acid specific extraction methods discussed herein. Pathogens may be distinguished from other pathogens based on their specific polynucleotide sequences. Specific pathogens have specific polynucleotide sequences that are not found in other pathogens. Nucleic acid specific to different strains of the same pathogen may be detected by sequence specific fashion.

Genetically Modified Organisms

The methods disclosed herein also allow nucleic acid from genetically modified organisms (GMOs) to be prepared for detection. Examples of GMOs include, but are not limited to, organisms in which one or more genes have been modified, added, or deleted. GMOs may be characterized by the presence of one or more specific genes, absence of one or more specific genes, specific alteration, or altered expression of one or more specific genes.

GMOs are frequently found in food samples. For example, genetically modified agricultural products, such as genetically modified grains, may be included in processed foods containing large quantities of polysaccharides. In order to prepare nucleic acid specific to the genetically modified organisms, glycosidase is provided to a food sample according to the methods disclosed herein. Nucleic acid of the GMO, which are frequently present in low quantities, may then be detected.

Non-Indigenous Fluora and Faunza

The methods disclosed herein also provide a method for preparing nucleic acid specific to non-indigenous fluora and fauna. Organisms that are not indigenous to a particular region present environmental and biological hazards to indigenous fluora and fauna. The presence of non-indigenous fluora and fauna frequently contains polysaccharides often in high quantities. The presence and number of non-indigenous fluora and fauna may be measured using the methods of the reaction.

As another example, food samples may also contain game meat that is killed out of season, or is obtained from endangered species. Such food samples may be identified based on nucleic acid sequences specific to the sex or species. The food samples also frequently contain polysaccharides, such as starch, that prevent nucleic acid from being readily detected. If sequence-specific extraction techniques are employed, the present methods allow nucleic acid specific to the sequence to be detected.

Kits

Kits for preparing nucleic acid from a polysaccharide-containing sample for detection are provided. The kit may include one or more glycosidases. In a further embodiment, the one or more glycosidases may include one or more glycoamylases. The kit may be formed to include such components as solvents and materials to particlize or solubilize a sample, additional solvents to remove other components of a sample, columns, and other components as disclosed herein. The kit can be packaged with instructions for use of the kit.

The reagents or reactants can be supplied in a solid form or dissolved/suspended in a liquid buffer suitable for inventory storage, and later for exchange or addition into the reaction medium when the test is performed. Suitable packaging is provided. The kit can optionally provide additional components used in the methods described above.

The kits can be employed to test a variety of biological samples, including body fluid, solid tissue samples, tissue cultures or cells derived therefrom and the progeny thereof, and sections or smears prepared from any of these sources. The kits may also be used to test a variety of samples such as surface matter, soil, water, agricultural and industrial samples, as well as samples obtained from food and dairy processing instruments, apparatus, equipment, disposable, and non-disposable items.

### EXAMPLES

The following non-limiting examples further illustrate the present application. It is readily apparent to those of ordinary skill in the art in light of the teachings of the present application that certain changes and modifications may be made thereto.

### Example 1

200 mg of ground corn was weighed and placed in a 2 ml microcentrifuge tube. 1 ml extraction buffer (10 mM Tris, 1 mM EDTA, 1% SDS, pH 7.5) was added. The sample was mixed well until no lumps were visible. The sample was heated in a 55° C. water bath for 10 minutes. The sample was then placed in a centrifuge for 4 minutes at 14,000 rpm. The upper aqueous phase was removed and placed in a new 1.5 ml tube.

Polysaccharides in the solution were then degraded by adding 50 ul Glycoamylase (1 U/ul in 10 mM acetate buffer), with incubation for 10 minutes at 55° C. 1/10 volume of 3 M potassium acetate (pH 4.8) solution was added, and mixed. Alternatively, a potassium acetate solution, pH 5.6, was used. The sample was centrifuged for 3 minutes at 14,000 rpm for 5 minutes. The liquid was removed without disturbing the pellet.

The supernatant was placed in a 0.5 ml column tube containing 70 mg of glass beads (Sigma G-9143, St. Louis, Mo.). The column was then centrifuged for 30 seconds at 2000 rpm. The flow through was discarded. The column was washed by adding 500 ul of 70% ethanol. Alternatively, 70% isopropanol may be used. The column was again centrifuged for 30 seconds at full speed, and the flow through was discarded. The wash process was repeated. The column was placed in a new collection tube spun 1 min. to remove any residual alcohol. The column was placed in a new 1.5 ml collection tube. 50 ul of TE pH 7.5 or water was added, and allowed to sit in the column for 1 minute at room temperature. The column was then centrifuged for 1 minute at full speed to elute the DNA. The DNA was ready for PCR.

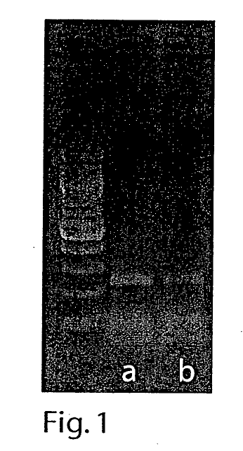

1-4 ul of eluted DNA was added to a PCR reaction. A gel of the PCR product is shown in FIG. 1a.

### Example 2

Nucleic acid in a maltodextrin sample were detected. 2 g of maltodextrin were added to a 50 ml tube. 3 mls of extraction buffer (10 mM Tris, 1 mM EDTA, 1% SDS, pH 7.5) were added and the sample was vortexed to remove lumps. The sample was then incubated in a water bath for 10 minutes at 55° C. Upon removal, the maltodextrin had solubilized and a clear viscous liquid was observed. The additional of 5 M NaCl to a concentration of greater than 2 M caused the maltodextrin to precipitate out of solution. The sample was placed on ice for 10 min.

Maltodextrin was removed by centrifugation. The supernatant was transferred to a fresh tube and the beads were added to the tube. The beads were allowed to equilibrate for ten minutes at room temperature to allowing nucleic acid binding. The tube was placed upright and the glass beads were sucked out of the tube and placed in a column. The column was washed by adding 500 ul of 70% ethanol. Alternatively, 70% isopropanol was used. The column was again centrifuged for 30 seconds at full speed, and the flow through was discarded. The wash process was repeated. The column was placed in a new collection tube spun 1 min. to remove any residual alcohol. The column was placed in a new 1.5 ml collection tube. 50 ul of TE pH 7.5 was added, and allowed to sit in the column for 1 minute at room temperature. The column was then centrifuged for 1 minute at full speed to elute the DNA.

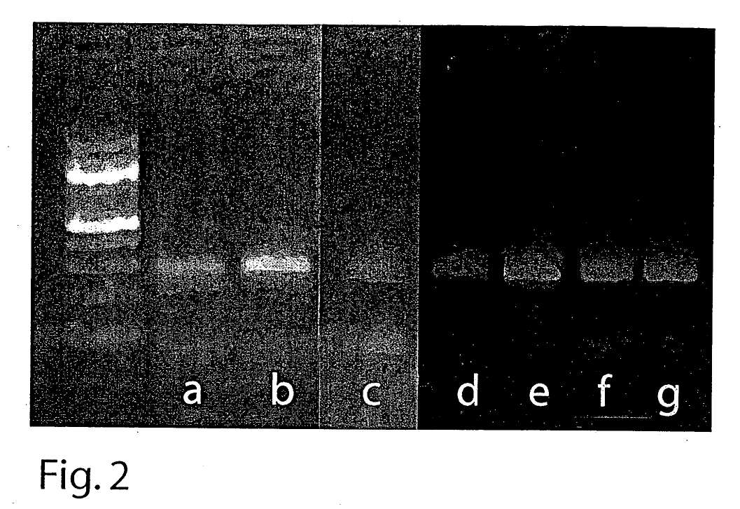

The DNA was detected by PCR. Generally 1-4 ul of eluted DNA is used in a PCR reaction. The amplification product is depicted in FIG. 2.

### Example 3

For the isolation of bacteria in a starch sample, buffer conditions are modified to utilize different surfactants such as CTAB, Trition X, or Tween all at concentrations, between 1%-10%. Different salts such as NaCl, Potassium acetate are used at different stages to aid in cell lysis. Alternatively low speed centrifugation is used to remove excess starch product from the sample to make isolation of the bacterial easier. Once most of the starch is removed, heat is used to aid in cell lysis. Upon the removal of most of the starch product and lysis of the bacteria, 1/10 volume of 3 M potassium acetate, pH 4.8 solution is added, and mixed. Alternatively potassium acetate solution at pH of 5.6 can be used. The sample is then centrifuged for 3 minutes at 14000 rpm for 5 minutes. The liquid is removed without disturbing the pellet.

The supernatant is placed in a 0.5 ml column tube with glass beads (Sigma G-9143). The column is then centrifuged for 30 seconds at 2,000 rpm. The flow through is discarded. The column is washed by adding 500 ul of 70% ethanol. Alternatively, 70% isopropanol may be used. The column is again centrifuged for 30 seconds at full speed, and the flow through is discarded. The wash process is repeated. The column is placed in a new collection tube spun 1 min. at full speed to remove any residual alcohol. The column is placed in a new 1.5 ml collection tube. 50 ul of TE pH 7.5 was added, and allowed to sit in the column for 1 minute at room temperature. The column is then centrifuged for 1 minute at full speed to elute the DNA. The DNA is in condition for PCR.

Generally 1-4 ul of eluted DNA is used in a PCR reaction.

### Example 4

This example illustrates that the methods disclosed herein were used to prepare nucleic acid from one gram of polysaccharide-containing sample.

The following kit components were stored at room temperature: 175 mL Buffer 1; 3.5 mL of Buffer 2; 17.5 mL Buffer 3; 3.2 mL Buffer 4; 5 tubes each of Reagent A, 50 columns (containing two glass fiber disks (Whatman GF-D, Houston, Tex.) and collection tubes, and 50 elution tubes. Buffer 1 was 10 mM Tris HCL pH 7.5, 1 mM EDTA, 1% SDS. Buffer 2 was 10 mM sodium acetate buffer, pH 4.5. Buffer 3 was 3 M potassium acetate solution (60 ml 5 M potassium acetate, 10 ml glacial acetic acid, 30 ml water, pH 5.6). Alternatively Buffer 3 was (60 ml 5 M potassium acetate, 11.5 ml glacial acetic acid, 28.5 ml water, pH 5.6). Buffer 4 was 10 mM Tris HCL pH 7.3. Reagent A was powdered glycoamylase (to be Glycoamylase 1 U/ul once the sodium acetate solution is added).

Polysaccharide-containing samples were mixed well with 2.8 mL or up to 3.0 mL of Buffer 1. Alternatively, additional buffer 1 was added to fully hydrate and liquefy the sample.

To test a sample's hydration point, a pre-hydration test was conducted by measuring 1 g of a sample and determining the quantity of water needed to hydrate and liquefy the sample. Once this was determined the same amount of buffer was then used to hydrate an analogous sample. The optimum amount of lysis buffer recovery after the first centrifugation step was between 600-800 μL. Alternatively the optimal amount of lysis buffer recovery after the first centrifugation step was all that could be recovered.

Reagent A was prepared. 650 μL of Buffer 2 was added to the vial labeled Reagent A. The mixture was mixed, but not vortexed. The hydrated reagent A was stored at −20° C. Care was taken to avoid repeated freeze and thaw. Unhydrated Reagent vials were stable at room temperature. Five vials of Reagent A were supplied, each capable of performing 10 extractions.

1 gram of a sample suspected of containing nucleic acid was ground and place it in a 15 mL tube. 2.8 to 3.0 mL of Buffer 1 was added to the sample tube. The contents of the tube were mixed well to avoid lumps. Thorough hydration of the sample was confirmed. If additional dry sample remained in the solution or the sample resembled paste, more Buffer 1 was added in 1 ml increments, and mixed well.

The mixture was placed on a 55° C. water bath for 10 min. Subsequently, the sample was placed in the centrifuge and spun for 10 min at maximum speed (for the centrifuge and tubes). Up to 800 μL of the supernatant was removed. Some supernatant remained in the tube. The removed supernatant was placed in a new 1.5 mL tube, and pellet carryover was limited. Alternatively all the supernatant was removed.

50 μL of the Reagent A solution was added. After mixing, the mixture was incubated for 10 min at 55° C. 0.3 volumes of Buffer 3 were added. The sample was chilled to between 0° C. and −20° C. The solution was allowed to sit for 1 to 5 min. The sample was centrifuged the sample 5 min at 14,000×g. The liquid was removed without disturbing the pellet and placed in a fresh 2.0 ml tube.

0.5-0.8 volumes of 95% ethanol were added to the liquid, and the components were mixed by inversion. The sample was centrifuged 1 min at 14,000×g to pellet any precipitate. 900 μL of the supernatant was placed in a column tube. The liquid immediately activated the glass bead complex (glass bead clumped together by using a 25 mM sucrose solution with dye and allowing the beads to dry in the column) and caused a color change and dissociation to occur from green to clear. The sample was centrifuged for 30 seconds at 2,000×g. The column flow through was discarded and the column was returned to the collection tube.

Up to 900 μL of the remaining supernatant was added to the column tube. The tube was centrifuged for 30 seconds at 2,000×g. The flow through was discarded and the column was returned to the collection tube.

The column was washed by adding 400 μL of 70% ethanol. The column was centrifuged for 30 sec at 14,000×g, the flow through was discarded, and the column was returned to the collection tube. (alternatively 70% isopropanol, was used.) The wash was repeated, the flow through was discarded, and the column returned to the collection tube. The column was spun 1 min at 14,000×g to remove any residual alcohol, and placed in a clean 1.5 mL elution tube.

50-80 μL of Buffer 4 was added, and the column was equilibrated at room temperature for 1 to 5 minutes. For improved yield, buffer 4 was pre-warmed to 55° C., or the sample can incubate at 55° C. TE buffer (for longer storage) or water (prior to sequencing applications) was added. Alternatively 10 mM Tris was added. The column was spun for 1 min at 14,000×g to elute the DNA. As an alternative, all centrifugation of microfuge tubes were accomplished at 6,000 rpm. The time of centrifugation times were increased accordingly.

The DNA was detected by PCR. Generally 1-4 μL of eluted DNA was used in a PCR reaction.

An agarose gel of PCR amplicons derived from nucleic acid obtained by the above methods is shown in FIG. 2. The amplified nucleic acid corresponds to a portion of the rubisco gene amplified from nucleic acid extracted from 2a) maltodextrin and 2b) wheat flour. FIG. 2c shows the amplified nucleic acid that corresponds to a portion of the rubisco gene amplified from nucleic acid extracted from corn chips. FIGS. 2d and 2e show the amplified nucleic acid corresponds to a portion of the rubisco gene amplified from nucleic acid extracted from corn meal and soy flour, respectively.

An agarose gel of PCR amplicons derived from nucleic acid obtained by the method is disclosed in FIG. 2. The amplified nucleic acid corresponds to a portion of the rubisco gene amplified from nucleic acid extracted from 2f) corn kernel and 2g) papaya fruit.

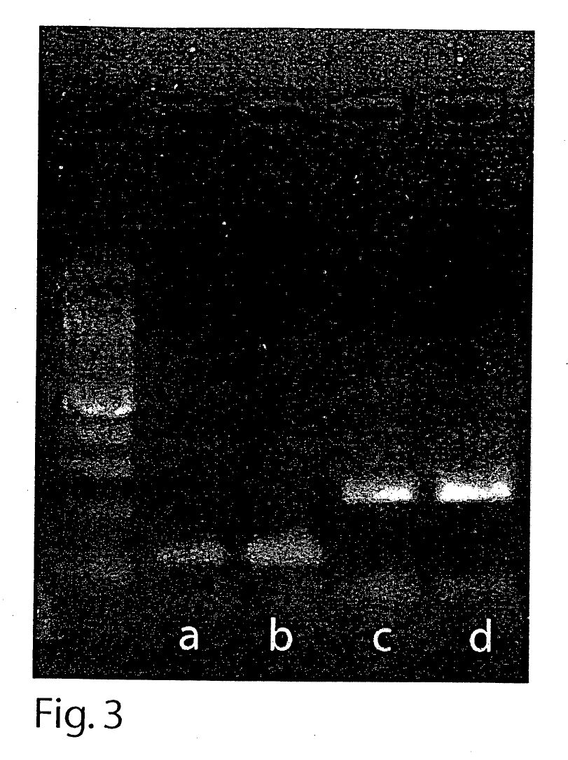

Another agarose gel of PCR amplicons derived from nucleic acids obtained by the method is disclosed in FIG. 3. The primers used in the amplification reaction corresponded to SEQ ID NOS: 3 and 4. The amplified nucleic acid corresponds to a portion of the lectin gene amplified from nucleic acid extracted from 3a) soy meal and 3b) soy flour, and a portion of the rubisco gene amplified from nucleic acid extracted from 3c) corn meal, and 3d) corn flour.

### Example 5

This example illustrates that the methods disclosed herein were used to prepare nucleic acid from 0.2 gram of polysaccharide-containing sample.

The following kit components were stored at room temperature: 91.0 mL Buffer 1; 3.5 mL Buffer 2; 14.0 mL Buffer 3; 3.2 mL Buffer 4, 5 aliquots Reagent A, 50 columns (containing two glass fiber disks) and associated collection tubes, and 50 elution tubes. Buffer 1 was 10 mM Tris HCL pH 7.5, 1 mM EDTA, 1% SDS. Buffer 2 was 10 mM sodium acetate buffer, pH 4.5. Buffer 3 was 3 M potassium acetate solution (60 mL 5 M potassium acetate, 10 mL glacial acetic acid, 30 mL water, pH 5.6). Alternatively Buffer 3 was (60 mL 5 M potassium acetate plus 11.5 mL glacial acetic acid, 28.5 mL water, pH 5.6). Buffer 4 was 10 mM Tris HCL pH 7.3. Reagent A was powdered glycoamylase (which was glycoamylase 1 U/ul once the sodium acetate solution was added).

In general, most of starch-like samples mixed well with 1 mL of Buffer 1. In some cases however, more buffer was needed to fully hydrate and liquefy the sample. If needed, up to 1.4 mL was added to hydrate a sample.

A pre-hydration test was done by simply measuring out 0.2 grams of a sample and determining the quantity of much water needed to hydrate and liquefy the sample. Once this was determined, the same amount of buffer was then used to hydrate an analogous sample.

For 0.2 gram samples, Reagent A was prepared as follows. 650 μL of Buffer 2 was added to the vial labeled Reagent A. The mixture was mixed, but not vortexed. The hydrated reagent A was stored at −20° C. Care was taken to avoid repeated freeze and thaw. Unhydrated Reagent A vials were stable at room temperature. Five vials of Reagent A were supplied, each capable of performing 10 extractions.

0.2 grams of a sample suspected of containing nucleic acid was ground and place it in a 2 mL tube. 1 mL of Buffer 1 was added to the sample tube. The contents of the tube were mixed well to avoid lumps.

The mixture was placed on a 55° C. water bath for 10 min. Subsequently, the sample was placed in the centrifuge and spun for 4 minutes at 14,000 rpm. The supernatant was placed in a new 1.5 mL tube, and pellet carryover was limited. Alternatively all the supernatant was removed.

50 μL of the Reagent A solution was added (or for comparison was added without the enzyme). After mixing, the mixture was incubated for 10 min at 55° C. 0.3 volumes of Buffer 3 were added. The sample was chilled to between 0° C. and 20° C. (The sample can also be stored at these temperatures.) The solution was allowed to sit for 1 to 5 min. The sample was centrifuged for 5 min at 14,000 rpm. The liquid was removed without disturbing the pellet and place it in a fresh 2.0 ml tube.

0.5-0.8 volumes of 95% ethanol were added to the liquid, and the components were mixed by inversion. (Alternatively, 95% isopropanol was used as a substitute.) The sample was centrifuged 1 min at 14,000 rpm to pellet any precipitate. 900 μL of the supernatant was placed in a column tube. The liquid immediately activated the glass bead complex and caused a color change and dissociation to occur from green to clear. The sample was centrifuged for 30 seconds at 2,000 rpm. The column flow through was discarded and the column was returned to the collection tube.

900 μL of the remaining supernatant was added to the column tube. The tube was centrifuged for 30 seconds at 2,000 rpm. The flow through was discarded column was returned to the collection tube. The centrifugation process was repeated.

The column was washed by adding 400 μL of 70% ethanol. The column was centrifuged for 30 sec at 14,000 rpm, the flow through was discarded, and the column was returned to the collection tube. (70% isopropanol can be used as an alternative to 70% ethanol.) Alternatively, the sample stored before or after the addition of ethanol. The wash was repeated, the flow through was discarded, and returned column to the collection tube. The column was spun 1 min at 14,000 rpm to remove any residual alcohol, and placed in a 1.5 mL elution tube.

50 μL of Buffer 4 was added, and the column was equilibrated at room temperature for 1 to 5 minutes. For improved yield, buffer 4 can be prewarmed to 55° C., or the sample can incubate at 55° C. Alternatively, TE buffer (for longer storage) water (prior to sequencing applications) was added. The column was spun for 1 min at 14,000 rpm to elute the DNA.

As an alternative, all centrifugation of microfuge tubes were conducted at 6,000 rpm. The time of centrifugation times were increased accordingly.

The DNA was detected by PCR. Generally 1-4 μL of eluted DNA was used in a PCR reaction. All PCR reaction were done using primers specific for rubisco (SEQ ID NOS: 5 and 6).

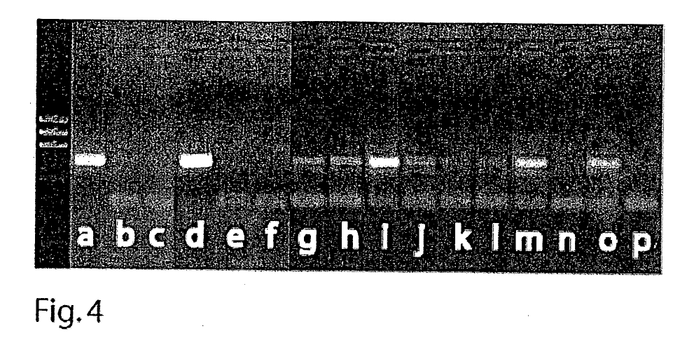

FIG. 4 depicts an agarose gel of PCR amplicons generated by PCR using primers specific to the rubisco gene for different samples.

Processed foods high in polysaccharides and lower in nucleic acid content were detected by PCR when using glycoamylase. Specifically, gel lane ‘b’ in FIG. 4 shows a rubisco PCR amplicon from a sample of corn chips when the sample is treated with glycoamylase. No PCR amplicon was observed in gel lane ‘e’ when the sample was not treated with glycoamylase. Similarly, gel lane ‘c’ in FIG. 4 shows a rubisco PCR amplicon from a sample of corn starch when the sample is treated with glycoamylase. No PCR amplicon was observed in lane ‘f’ when the sample is not treated with glycoamylase.

Lane ‘g’ in FIG. 4 shows a rubisco PCR amplicon from a sample of Twix® cookies when the sample was treated with glycoamylase. Only a very faint PCR amplicon was observed in lane ‘k’ when the sample was not treated with glycoamylase. Likewise lane ‘h’ in FIG. 4 shows a rubisco PCR amplicon from a sample of ground wheat crackers treated with glycoamylase. Again, only a very faint PCR amplicon was observed in lane ‘l’ when the sample was not treated with glycoamylase. Lane ‘i’ in FIG. 4 shows a rubisco PCR amplicon from a sample of miso powder when the sample was treated with glycoamylase. Again, only a very faint PCR amplicon was observed when the sample was not treated with glycoamylase. Finally, lane ‘j’ shows a rubisco PCR amplicon from a sample of oat cereal when the sample was treated with glycoamylase. No PCR amplicon was observed in lane ‘n’ when the sample was not treated with glycoamylase.

Processed food samples having small quantities of nucleic acid and large quantities of polysaccharide were prepared for detection by treating with glycoamylase (a glycosidase). After preparation of nucleic acid by providing glycoamylase in each processed food sample, nucleic acids were readily detected. In the absence of glycoamylase, the nucleic acids of the processed food samples were either undetectable or only faintly detectable.

Lane ‘a’ in FIG. 4 shows a rubisco PCR amplicon from a sample of ground seeds when the sample was treated with glycoamylase. It is noted that the presence of a large amount of nucleic acid in the seed sample and lack of extensive food processing likely explains the detection of the amplicon after amplification by PCR.

### Example 6

This example shows preparing nucleic acid from a 1 gram food sample.

This protocol demonstrates scalability of DNA extraction, the use of columns, and the use of ethanol and increased potassium acetate to enhance and the use of chilling to enhance the removal of starch from the sample.

The following buffers were prepared. Buffer 1 was 10 mM Tris, 1 mM EDTA, 1% SDS. Buffer 3 was 5 M potassium acetate. Buffer 4 was 10 mM Tris pH 7.5. Buffer 5 was 10 mM sodium acetate buffer, pH 4.5. Reagent 6 was amyloglucosidase enzyme. The columns contained 2 disks of matted glass fiber.

Buffer 2 was prepared before first use and stored at −20° C. Buffer 2, was made by adding 150 ul of buffer 5 to the vial labeled reagent 6. The hydrated solution was centrifuged for 20 seconds at 13,000-16,000 rpm. The upper phase was transformed to the supplied tube labeled buffer.

One gram of ground corn was placed in a 15 ml tube. 2 ml of buffer 1 was added and the sample was mixed on a vortexer. The sample was placed in a 55° C. water bath for 10 min. After incubation the sample was placed in swinging bucket centrifuge and spun for 10 min at 3,400×g. The clarified supernatant was removed and transferred to a 2 ml tube. 50 uL of buffer 2 was added the tube was mixed and incubated for 10 minutes at 55° C. The sample is allowed to cool and 0.3 volumes (of the supernatant) of buffer 3 are added and mixed. The sample was placed on ice for 5 minutes and then centrifuged for 5 minutes at full speed (14,000 rpm). The clarified supernatant was removed and transferred to a fresh 2 ml tube. 0.5 volumes of 95% ethanol was added and the tube was mixed by inversion. 900 μL of the mixed supernatant was added to the column (inside a collection tube) and the sample was centrifuged for 30 seconds at 2,000 rpm. The column was removed and the flowthrough discarded. The column was returned to the collection tube and the remaining supernatant was added to the column. Again the sample was centrifuged for 30 seconds at 2,000 rpm and the flowthrough discarded. The column was washed twice by adding 400 μL of 70% ethanol and centrifuged for 30 sec at 10,000 rpm. The flowthrough was discarded and the column returned to the collection tube. Residual alcohol was removed by a final spin for 1 minute at 10,000 rpm. The column was transfered to a fresh 1.5 mL tube and 80 μL of Buffer 4 was added to the column. The sample was left to stand 5 minutes and the DNA was finally eluted by centrifugation for 1 minute. 1-4 uL of sample was removed and added to a freshly made PCR reaction mixture that included 2.5 ul 10× PCR buffer, 1.5 ul MgCl 50 mM, 0.5 ul dNTP 10 mM, 0.25 ul BSA, 0.25 ul Taq, 0.25 ul of each primer, 17.5 ul water, and 2 ul of sample.

The forward primer of the corn samples was CCGCTGTATCACAAGGGCTGGTACC (SEQ ID NO:1), and the reverse primer was GGAGCCCGTGTAGAGCATGACGATC (SEQ ID NO: 2). The primers correspond to the invertase gene.

The positive control PCR reaction was spiked corn DNA and primers specific for the invertase gene. The negative DNA control PCR reaction contained primers specific for the invertase gene but had no corn DNA. Reactions were run on an MJ Research PCT-100 machine according to the following conditions. 95° C. initial melt for 2 minutes, followed by 42 cycles of 95° C. for 20 sec, 53° C. for 10 sec and 72° C. for 10 sec with a final step at 72° C. for 3 min and a hold at 4° C.

After PCR, the samples were run on a 2% TBEE agarose gel for 30 min at 100V. Gels were then transferred to a UV transluminator and photographed with a Polaroid Land camera. Amplified invertase sequence was detected for the positive control and test sample, but not in the negative control.

All publications, patents, and patent applications cited herein are hereby incorporated by reference in their entirety for all purposes to the same extent as if each individual publication, patent, or patent application were specifically and individually indicated to be so incorporated by reference. Although the foregoing has been described in some detail by way of illustration and example for purposes of clarity of understanding, it is readily apparent to those of ordinary skill in the art in light of the teachings of the present application that certain changes and modifications may be made thereto without departing from the spirit and scope of the claims.

Applicants have not abandoned or dedicated to the public any unclaimed subject matter.

1. A method of preparing nucleic acid from a polysaccharide-containing sample, said method comprising:
providing one or more glycosidases to the polysaccharide-containing sample, thereby generating a glycosidase-treated sample.

2. The method of claim 1, further comprising extracting said nucleic acid from the glycosidase-treated sample.

3. A method of detecting nucleic acid in a polysaccharide-containing sample, comprising:
preparing the nucleic acid according to the method of claim 1; and
detecting the nucleic acid.

4. The method of claim 1, wherein the one or more glycosidases includes a glycosidase selected from the group consisting of a glycoamylase, a debranching enzyme, a heterosaccharide degrading enzyme, and a non-glucose homosaccharide degrading enzyme.

5. (canceled)

6. The method of claim 4, wherein the glycoamylase is selected from the group consisting of an alpha-amylase, a beta-amylase, a glucan alpha 1,4-glucosidase, a glucan alpha 1,4-glucosidase, and a glucan alpha 1,6-glucosidase.

7-11. (canceled)

12. The method of claim 2, wherein said extracting includes providing an alcohol to the sample.

13-15. (canceled)

16. The method of claim 1, wherein the polysaccharide-containing sample comprises starch.

17-18. (canceled)

19. The method of claim 1, wherein the polysaccharide-containing sample includes one or more components selected from the group consisting of corn meal, soy flour, wheat flour, corn starch, corn chips, and maltodextrin.

20. The method of claim 1, further comprising removing the polysaccharides from the glycosidase-treated sample.

21. The method of claim 1, further comprising removing cellular components from the sample.

22-23. (canceled)

24. The method of claim 21, wherein the cellular components are removed by providing to the sample one or more salts selected from the group consisting of potassium acetate and sodium acetate, thereby precipitating said cellular components.

25. The method of claim 21, wherein said extracting includes applying the sample to a column.

26. (canceled)

27. A kit for preparing nucleic acid from a sample comprising:
a) a glycosidase; and
b) instructions for using the kit.

28. The kit of claim 27, wherein the glycosidase is selected from the group consisting of a glycoamylase, an alpha-amylase, a beta-amylase, a glucan alpha 1,4-glucosidase, and a glucan alpha 1,6-glucosidase.

29-32. (canceled)

33. The kit of claim 27, further comprising one or more salts selected from the group consisting of potassium acetate and sodium acetate.

34. The kit of claim 27, further comprising sodium dodecyl sulfate (SDS).

35. The kit of claim 27, further comprising an alcohol.

36. (canceled)

37. The kit of claim 27, further comprising a column.

38. The kit of claim 37, wherein the column contains glass beads or glass wool.

39. (canceled)

40. The method of claim 3, wherein detecting the nucleic acid includes amplification thereof.


SEQUENCE LISTING (6 sequences)

SEQ ID NO: 1 - DNA, 25 bp, Artificial
  primer
ccgctgtatcacaagggctggtacc

SEQ ID NO: 2 - DNA, 25 bp, Artificial
  primer
ggagcccgtgtagagcatgacgatc

SEQ ID NO: 3 - DNA, 20 bp, Artificial
  primer
gacgctattgtgacctcctc

SEQ ID NO: 4 - DNA, 25 bp, Artificial
  primer
gaaagtgtcaagcttaacagcgacg

SEQ ID NO: 5 - DNA, 23 bp, Artificial
  primer
tctgttactaacatgtttacttc

SEQ ID NO: 6 - DNA, 26 bp, Artificial
  primer
cccaatttaggtttaatagtacatcc